SEQUENCE LISTING (2 sequences)

SEQ ID NO: 1 - DNA, 34 bp, Artificial Sequence
  Description of Artificial Sequence Primer
tggaattctttgcggaatattgcctcagttttgg

SEQ ID NO: 2 - DNA, 45 bp, Artificial Sequence
  Description of Artificial Sequence Synthetic oligonucleotide
tttggatccttatttaattgtcccagcgtcaagtaatggaaaggg

(19) United States
(12) Patent Application Publication
(10) Pub. No.: US 2005/0095651 A1
(43) Pub. Date: May 5, 2005

(54) Photoswitchable method for the ordered attachment of proteins to surfaces

(75) Inventors: Julio A. Camarero, Livermore, CA (US); James J. De Yoreo, Clayton, CA (US); Youngeun Kwon, Livermore, CA (US)

Correspondence Address: Ann M. Lee;Assistant Laboratory Counsel, Lawrence Livermore National Laboratory, P.O. Box 808, L-703, Livermore, CA, 94551, US

(73) Assignee: The Regents of the University of California

(21) Appl. No.: 10/918,759

(22) Filed: Aug. 12, 2004

Related U.S. Application Data
(60) Provisional application No. 60/494,675, filed on Aug. 12, 2003.

(51) Int. Cl. G01N 33/53; C07K 14/47

(52) U.S. Cl. 435/7.1; 530/396

(57) ABSTRACT

Disclosed herein is an improved method for the attachment of proteins to any solid support with control over the orientation of the attachment. The method is extremely efficient, not requiring the previous purification of the protein to be attached, and can be activated by UV-light. Spatially addressable arrays of multiple protein components can be generated by using standard photolithographic techniques.

### CLAIM OF PRIORITY IN PROVISIONAL APPLICATION

This application is related to Provisional Application No. 60/494,675 filed Aug. 12, 2003 entitled “Chemoenzymatic-like and Photoswitchable Method for the Ordered Attachment of Proteins to Surfaces”, and claims priority thereto under 35 USC 120. Provisional Application No. 60/494,675 is herein incorporated by reference in its entirety.

The United States Government has rights in this invention pursuant to Contract No. W-7405-ENG-48 between the United States Department of Energy and the University of California for the operation of Lawrence Livermore National Laboratory.

### BACKGROUND

Various methods are available for attaching proteins to solid surfaces. Most rely on either (1) non-specific adsorption, or (2) the reaction of chemical groups within proteins (e.g., amino and carboxylic acid groups) with surfaces containing complementary reactive groups. In both cases the protein is attached to the surface in random orientations. The use of recombinant affinity tags addresses the orientation issue, but the interactions of the tags are often reversible. Therefore, the recombinant affinity tags require large mediator proteins in order to remain stable over the course of subsequent assays.

References:

1. S. Fields, Proteomics. Proteomics in genomeland, Science 291(5507), 1221-4. (2001).
2. H. Zhu et al., Protein arrays and microarrays, Curr Opin Chem Biol 5(1), 40-5. (2001).
3. G. Wu et al., Bioassay of prostate-specific antigen (PSA) using microcantilevers, Nat Biotechnol 19(9), 856-60. (2001).
4. H. Zhu et al., Analysis of yeast protein kinases using protein chips, Nat Genet 26(3), 283-9. (2000).
5. H. Zhu et al., Global analysis of protein activities using proteome chips, Science 293(5537), 2101-5. (2001).
6. D. L. Wilson et al., Surface organization and nanopatterning of collagen by dip-pen nanolithography, Proc Natl Acad Sci U S A 98(24), 13660-4. (2001).
7. K. B. Lee et al., Protein nanoarrays generated by dip-pen nanolithography, Science 295(5560), 1702-5. (2002).

### DETAILED DESCRIPTION

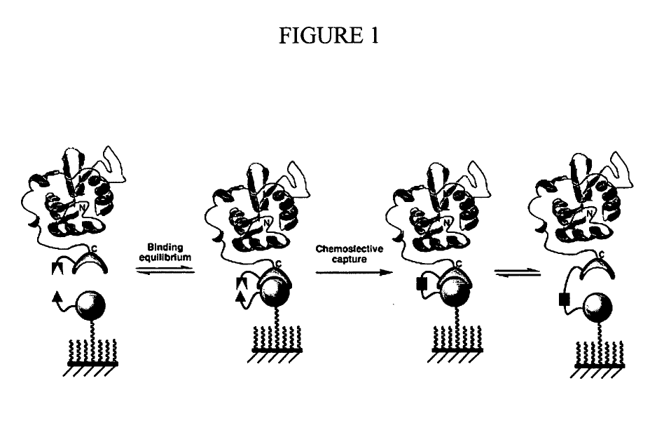

Methods for the chemoselective attachment of proteins to surfaces has been developed. (See J. A. Camarero, “Chemoselective Ligation Methods for the Ordered Attachment of Proteins to Surfaces”, in Solid-fluid Interfaces to Nanostructural Engineering, J. J. de Yoreo, Editor. 2004, Plenum/Kluwer Academic Publisher: New York and C. L. Cheung et al., Fabrication of Assembled Virus Nanostructures on Templates of Chemoselective Linkers Formed by Scanning Probe Nanolithography, J. Am. Chem. Soc. 125, p. 6848, 2003.) These methods rely on the introduction of two unique and mutually reactive groups on the protein and the support surface. The reaction between these two groups usually gives rise to the selective attachment of the protein to the surface with total control over the orientation. However, these methods, although highly selective, rely on uncatalyzed pseudo-bimolecular reactions with little or no entropic activation at all. This lack of entropic activation means that the efficiency of these bimolecular-like reactions will depend strongly on the concentration of the reagents (i.e., the protein to be attached). A way to overcome this intrinsic entropic barrier and make attachment reactions even more efficient and selective, even under high dilution conditions, is through the use of a highly selective molecular recognition event to bring together the two reactive species. This event will increase dramatically the local effective concentration of both reacting species thus accelerating the corresponding attachment reaction even under unfavorable conditions (i.e., low concentration and even in the presence of other proteins). Referring to FIG. 1, this entropic activation approach can also be used to improve the efficiency and rate of attachment of proteins to surfaces with total control over the orientation of the attachment. Considerably less protein is required since the ligation reaction works very efficiently even under high dilution conditions. There is no need for purification since at high dilution the only protein that will react with the surface will be the one having the complementary affinity and reactive tag. The introduction of complementary moieties in the protein and the surface form a stable and specific intermolecular complex. Once formed, this complex can permit a selective reaction of the complementary chemical groups leading to the covalent attachment of the protein to the surface.

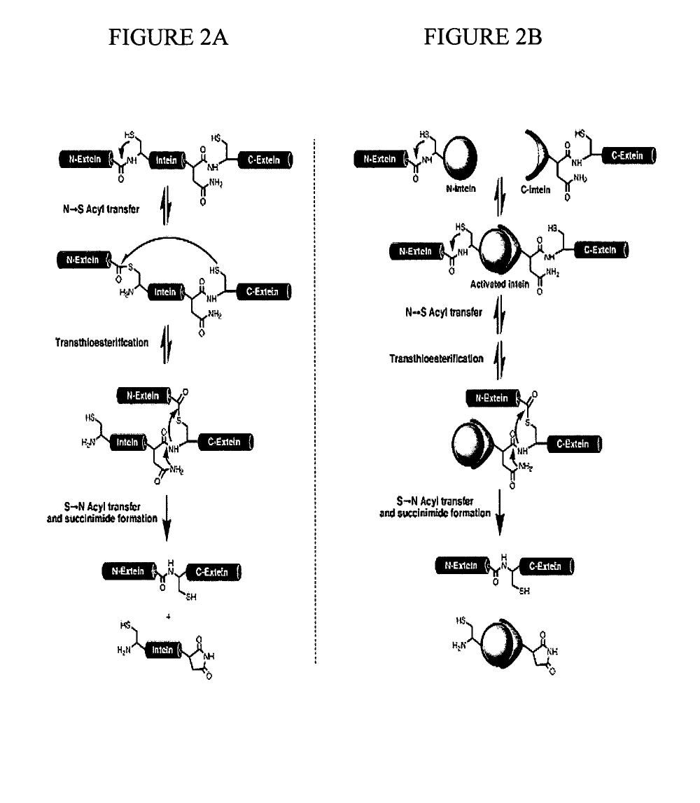

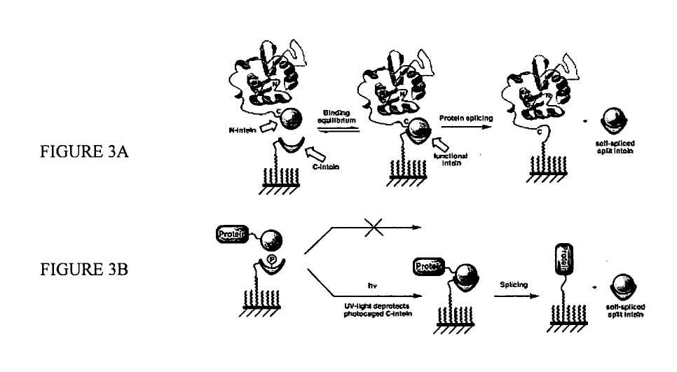

Disclosed herein is a photo-switchable method for the selective attachment of proteins through the C-terminus. The method is based on the protein trans-splicing process as shown in FIG. 2B. This process is similar to the protein splicing disclosed by Xu in (insert ref. 1), which is shown in FIG. 2A, however, in the method disclosed herein, the intein self-processing domain is split in two fragments (called N-intein and C-intein, respectively). These two intein fragments alone are inactive, however, when they are put together under the appropriate conditions they bind specifically to each other yielding a totally functional splicing domain, which splices itself out at the same time both extein sequences are ligated. In the method disclosed herein, one of the fragments (C-intein) will be covalently attached to the surface through a small peptide-linker while the other fragment (N-intein) will be fused to the C-terminus of the protein to be attached. When both intein fragments interact, they will form the active intein which ligates the protein of interest to the surface at the same time the split intein is spliced out into solution. Referring to FIG. 3A, the C-intein fragment is attached to the surface and the N-intein fragment is fused to the C-terminus of the protein to be attached. When this fusion protein is exposed to a C-intein-containing surface, the two intein fragments associate yielding a fully operational intein domain that then splices out at the same time attaching the protein to the surface.

The split DnaE intein from Synechocystis sp. PCC6803 is a naturally occurring split intein that was first discovered by Liu and co-workers H. Wu et al., Protein trans-splicing by a split intein encoded in a split DnaE gene of Synechocystis sp. PCC6803, Proc. Natl. Acad. Sci. USA 95, 9226-9231 (1998). It was also predicted through sequence analysis in an independent study by Gorbalenya. In contrast with other inteins engineered to act as trans-splicing elements, which only work after a refolding step, the C— and N-intein fragments of the DnaE intein are able to self-assemble spontaneously without any refolding step. The DnaE split intein comprises an N-intein fragment having 123 residues and a C-intein fragment of having only 37 residues. Referring to FIG. 3A, a recombinant fusion protein is expressed where the DnaE N-intein fragment is fused to the C-terminus of the protein to be attached to the surface. The C-intein fragment can be synthesized as a synthetic peptide by using a Solid-Phase Peptide Synthesis (SPPS) approach. This allows the introduction of an PEGylated alkylthiol moiety at the C-terminus of the C-intein peptide which is used for attachment to solid surfaces (e.g., gold or Si-based).

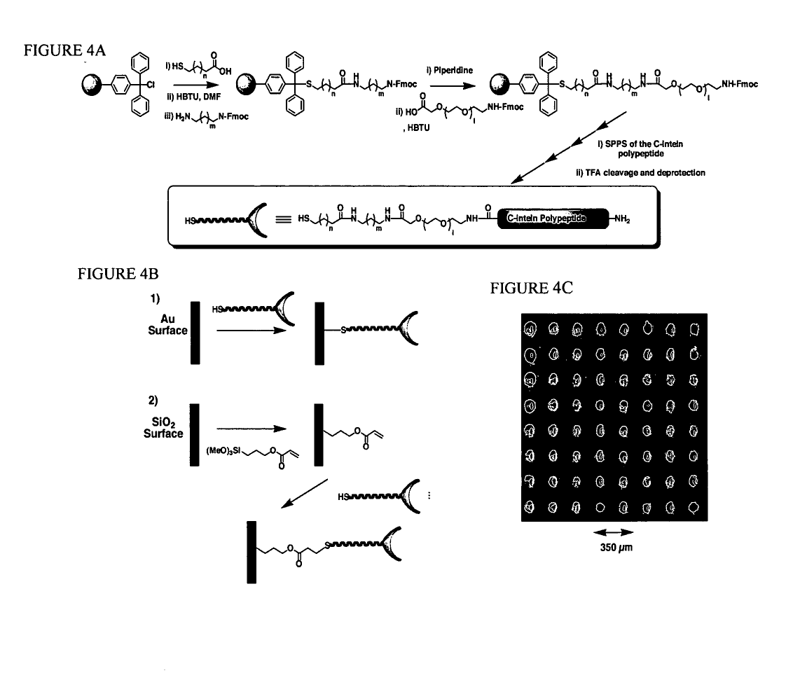

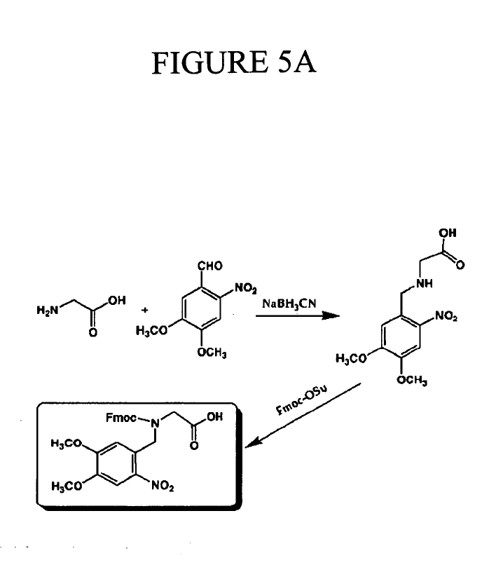

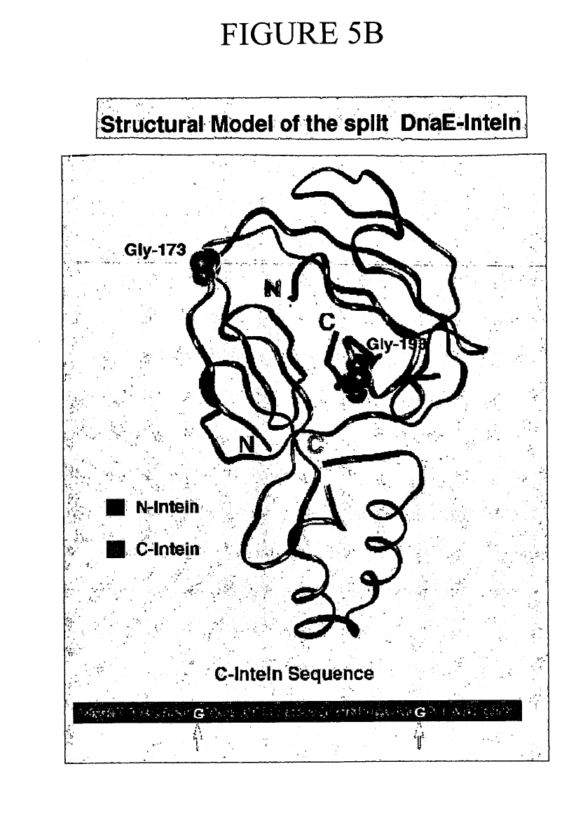

Spatially addressable protein arrays with multiple protein components can be created by photocaging. FIG. 5A shows a C-intein fragment where some of the functional side-chains or backbone amide groups key for the interaction with the N-intein are caged using a nitrobenzyl protecting group, such as the nitroveratryloxycarbonyl (Nvoc) or nitroveratryl (Nv). The Nv protecting group can be introduced into Gly, Ala, Asn, Gln and Lys residues to prevent the interactions between the two intein fragments as shown in FIG. 3B. For example, using the protecting group on the Gly residue 6, 11, 19 and/or 31 and/or Ala residues 29, 32, 34, and/or 35 is effective as is using the protecting group on the Asp residue 17 and/or 23, the Asn residues 25, 30 and/or 36, and/or the Gln residues 13 and/or 22. Removal of the group is achieved by exposure to UV-light (e.g., using a 10 μW pulse of 354-nm UV light generated from a He—Cd laser or similar source). When this photo-labile protecting group is removed by the action of UV-light, the two intein fragments assemble into a functional intein domain, thus allowing the attachment of the corresponding protein to the surface through protein splicing (See FIG. 3B). At the same time, both intein moieties are spliced out and consequently removed. FIG. 4A shows Fmoc-based solid-phase peptide synthesis of the C-intein on a PEGylated resin. After cleavage from the resin, the C-intein polypeptide is linked to its C-terminus through a PEGylated thiol linker. FIG. 4B shows that the linker serves as a spacer and can be used to chemoselectively attach the C-intein polypeptide to either gold or Si-based solid supports through its C-terminus. FIG. 4C is an epifluorescence image of a modified glass surface spotted with the C-intein polypeptide. After spotting, the glass slide was washed and incubated with a fluorescent dye which specifically reacted with the attached polypeptide. FIG. 5A shows the synthesis scheme of a backbone photocaged Gly residue for the solid-phase peptide synthesis of the photocaged C-intein. FIG. 5B is a structural model of the split DnaE-intein showing some of the Gly residues that can be photocaged in order to prevent the association of the C-intein and N-intein fragments.

Experimental

Materials and Methods.

Fmoc-amino acids, O-benzotriazol-1-yl-N,N,N′,N′-tetramethyluronium hexafluoro-phosphate (HBTU) and 4-Fmoc-hydrazine AM resin were obtained from Novabiochem. Methylene chloride (DCM), N,N-dimethylformamide (DMF) and HPLC-grade acetonitrile (MeCN) were purchased from Fisher. Trifluoroacetic acid (TFA) was purchased from Halocarbon. All other reagents were obtained from Aldrich Chemical Co. Analytical and semipreparative gradient HPLC were performed on a Hewlett-Packard 1100 series instrument with UV detection. Semipreparative HPLC was run on a Vydac C18 column (10 micron, 10×250 mm) at a flow rate of 5 mL/min. Analytical HPLC was performed on a Vydac C18 column (5 micron, 4.6×150 mm) at a flow rate of 1 mL/min. Preparative HPLC was performed on a Waters DeltaPrep 4000 system fitted with a Waters 486 tunable absorbance detector using a Vydac C18 column (15-20 micron, 50×250 mm) at a flow rate of 50 mL/min. All runs used linear gradients of 0.1% aqueous TFA (solvent A) vs. 90% MeCN plus 0.1% TFA (solvent B).¹H NMR spectra were obtained at room temperature on Bruker 400 MHz or Varian 90 MHz spectrometers. Electrospray mass spectrometric analysis was routinely applied to all synthetic peptides and components of reaction mixtures. ESMS was performed on a Applied Biosystems/Sciex API-150EX single quadrupole electrospray mass spectrometer. Calculated masses were obtained using the program ProMac 1.5.3.

Synthesis of PEGylated Thiol Linker Resin.

Trityl resin (1 g, 1.1 mmol/g) was swollen in DCM for 20 min and washed with dimethylformamide (DMF) and then dichloromethane (DCM). 3-Mercaptopropionic acid (2 mmol, 175 μL mg) in DCM:DMF (4 ml, 9:1 v/v) was added to the swollen resin. The reaction was kept for 18 h at room temperature with gentle agitation. The reacted resin was then washed with DCM and DMF. The carboxylic function of the resin was activated with 2-[1H-benzotriazolyl]-1,1,3,3-tetramethyluronium hexafluorophosphate (HBTU, 2 mmol) in DMF:DIEA (5 mL, 4:1 v/v) for 30 min. at room temperature. After washing with DMF, the activated resin was treated with mono-Fmoc-ethylenediamine hydrochloride (1.2 mmol, 383 mg) in DMF (4 mL) containing DIEA (1.5 mmol, 261 μL) for 2 h at room temperature. 200 mg of the N-Fmoc protected resin were then deprotected with 2% DBU and 20% piperidine in DMF solution. The resulting amino group was acylated with 3-[2-(2-{2-[2-(9H-fluoren-9-ylmethoxycarbonylamino)-ethoxy]-ethoxy}-ethoxy)-ethoxy]-propionic acid (0.21 mmol, 102 mg, Quanta Biodesign, Powell, Ohio) using HBTU (0.2 mmol) in DMF:DIEA (1 mL, 9:1 v/v) for overnight at room temperature with gentle agitation. The resin was then washed with DMF and DCM, dried under vacuum and stored until use.

Solid-Phase Peptide Synthesis of the C-Intein Polypeptides.

All peptides were manually synthesized using the HBTU activation protocol for Fmoc solid-phase peptide synthesis on the previously described resin. Coupling yields were monitored by the quantitative ninhydrin determination of residual free amine. Side-chain protection was employed as previously described for the Fmoc-protocol except for Fmoc-(1,2-dimethoxy-4-methyl-3-nitro-benzyl)-Gly-OH and Fmoc-Cys(StBu)-OH that were used to photocaged the corresponding Gly (residues 6, 11, 19 or 31) and to selectively protect Cys (residue 37), respectively.

### Synthesis of Fmoc-(1,2-dimethoxy-4-methyl-3-nitro-benzyl)-Gly-OH [Fmoc-(nitroveratryl)-Gly-OH]

The synthesis was performed as described in FIG. 4A. Briefly, 6-nitroveratraldehyde (111 mg, 1 mmol), H-Gly-OH.HCl (111.5 mg, 1 mmol) and NaBH3CN (126 mg, 1 mmol) were suspended in MeOH (15 mL) and stirred at 25° C. for 90 min. The suspension was concentrated to dryness in vacuo, and the residual oil was resuspended in dioxane-H₂O (1:1, 10 mL). Solid NaHCO₃ (0.26 g, 3 mmol) was added, the suspension was cooled in an ice bath, and Fmoc-OSu (0.5, 1.5 mmol) in dioxane (4 mL) was added. Stirring was continued for 90 min while cooling in an ice-bath and a 25° C. for another 90 min. The pH was adjusted to 9 by addition of solid NaHCO₃. The suspension was diluted with H₂O (40 mL) and washed with Et2O (2×50 mL). Phase separations were slow, and the organic layer remained cloudy. The aqueous layer was acidified to pH 3 with 4 M aqueous HCl and extracted with EtOAc (2×50 mL). The organic phases were pooled and concentrated to dryness in vacuo. The crude material was finally purified by preparative HPLC using a linear gradient of 15-100% solvent B over 30 min to give the desired Fmoc-(nitroveratryl)-Gly-OH (300 mg, 70% overall yield). The final product was characterized by RP—HPLC and ES-MS. ES-MS [observed mass=493.0±0.1 Da; calculated for C₂₆H₂₄N₂O₈=492.48 Da].

Functionalization of Glass Slides

This describes the procedure to produce the array shown in FIG. 4C. Plain glass micro-slides (VWR Scientific Products, USA) were cleaned with RCA solution (3% NH₃, 3% H₂O₂ in water) at 80° C. for 4 h. After thorough rinsing with deionized water, the slides were washed with MeOH and treated with a 2% solution of 3-acryloxypropyl trimethoxysilane (Gelest, Morrisville, Pa.) in MeOH containing 1% H₂O for 15 min. Before treating the slides, the silane solution was stirred for 10 min to allow the hydrolysis of the silane. After the silanization, the glass slides were washed with MeOH to remove excess silanol and dried under a N₂ stream. The adsorbed silane was then cured in the dark at room temperature under vacuum for 18 h. Standard microarray spotting techniques were used to attach proteins to modified glass slides in a microarray format. The different C-intein polypeptides were diluted in spotting buffer (50 mM sodium phosphate, 100 mM NaCl buffer at pH 7.5 containing 10% glycerol) at different concentrations (20 μM-500 μM) and arrayed in the acryloxy-containing glass slides using a robotic arrayer (Norgren Systems, Palo Alto, Calif., USA). C-Int polypeptides were spotted with a center-to-center spot distance of 350 μm with an average spot size of 200 μm in diameter. The slide was allowed to react for 18 h at room temperature. The unbound C-intein was washed. The unreacted acryloxy groups were capped using a solution of a PEGylated thiol. The bound C-intein was reacted with 5-IAF (a thiol-reactive fluorescein derivative) and then imaged using a ScanArray 5000 (488 nm laser).

Cloning and Expression of a MBP-N-Intein Fusion Protein.

The DNA encoding the DnaE N-intein (residues F771-K897) was isolated by PCR. The 5′ primer (5′-TG GAA TTC TTT GCG GAA TAT TGC CTC AGT TTT GG-3′) encoded a EcoRI restriction site. The 3′ oligonucleotide (5′- TTT GGA TCC TTA TTT AAT TGT CCC AGC GTC AAG TAA TGG AAA GGG-3′) introduced a stop codon as well as a BamHI restriction site. The PCR amplified N-Intein domain was purified, digested simultaneously with EcoRI and BamHI and then ligated into a EcoRI,BamHI-treated plasmid pMAL-c2 (New England Biolabs). The resulting plasmid pMAL-N-Intein was shown to be free of mutations in the N-Intein-encoding region by DNA sequencing. Two liters of E. coli BL21(DE3)pLysS⁺ cells transformed with pMAL-N-Intein plasmid were grown to mid-log phase (OD₆₀₀≈0.6) in Luria-Bertani (LB) medium and induced with 0.5 mM (isopropyl [symbol] -thiogalactopyranoside) IPTG at 37° C. for 4 h. The lysate was clarified by centrifugation at 14,000 rpm for 30 min. The clarified supernatant (ca. 40 mL) was incubated with 5 mL of maltose-beads (New England Biolabs), previously equilibrated with column buffer (0.1 mM EDTA, 50 mM sodium phosphate, 250 mM NaCl, 0.1% Triton X-100 at pH 7.2), at 4° C. for 30 min with gently shaking. The beads were extensively washed with column buffer (10×5 mL) and equilibrated with PBS (50 mM sodium phosphate, 100 mM NaCl at pH 7.2, 2×50 mL). The MBP-fusion protein adsorbed on the beads was then eluted with column buffer containing 20 mM maltose. The filtrates were pooled, and the protein was dialyzed and concentrated.

References

1. M.-Q. Xu et al., The mechanism of protein splicing and its modulation by mutation, EMBO J. 15(19), 5146-5153 (1996).
2. F. B. Perler, A natural example of protein trans-splicing, Trends Biochem Sci 24(6), 209-11. (1999).
3. H. Wu et al., Protein trans-splicing by a split intein encoded in a split DnaE gene of Synechocystis sp. PCC6803, Proc. Natl. Acad. Sci. USA 95, 9226-9231 (1998).
4. A. E. Gorbalenya, Non-canonical inteins, Nucleic Acids Res 26(7), 1741-8. (1998).
5. B. M. Lew et al., Protein splicing in vitro with a semisynthetic two-component minimal intein, J Biol Chem 273(26), 15887-90. (1998).
6. K. V. Mills et al., Protein splicing in trans by purified N— and C-terminal fragments of the Mycobacterium tuberculosis RecA intein, Proc Natl Acad Sci U S A 95(7), 3543-8. (1998).
7. T. C. Evans et al., Protein trans-splicing and cyclization by a naturally split intein from the dnaE gene of Synechocystis species PCC6803, J Biol Chem 275(13), 9091-4. (2000).
8. D. D. Martin et al., Jr., Characterization of a naturally occurring trans-splicing intein from Synechocystis sp. PCC6803, Biochemistry 40(5), 1393-402. (2001).
9. T. Vossmeyer et al., Combinatorial approaches toward patterning nanocrystals, J. Appl. Phys. 84(7), 3664-3670 (1998).
10. P. Roy et al., Local photorelease of caged thymosin b4 in locomoting keratocytes causes cell turning, J. Cell Biol. 153(5), 1035-1047 (2001).

All numbers expressing quantities of ingredients, constituents, reaction conditions, and so forth used in the specification and claims are to be understood as being modified in all instances by the term “about”. Notwithstanding that the numerical ranges and parameters setting forth the broad scope of the subject matter presented herein are approximations, the numerical values set forth in the specific examples are reported as precisely as possible. Any numerical value, however, inherently contain certain errors necessarily resulting from the standard deviation found in their respective testing measurements.

All publications and patents mentioned in the above specification are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in organic chemistry, biochemistry, molecular biology or related fields are intended to be within the scope of the following claims.

1. In a method for immobilizing a polypeptide to a surface using a split C-intein/N-intein, the improvement comprising:
creating a modified C-intein fragment, wherein at least one functional side-chain necessary for the interaction with the N-intein or at least one backbone amide group necessary for the interaction with the N-intein is caged using a 2-nitrobenzyl-based protecting group.

2. The method recited in claim 9, wherein at least one backbone amide group necessary for the interaction with the N-intein is a Gly residue or an Ala residue.

3. The method recited in claim 2, wherein the Gly residue is residue 6, 11, 19 and/or 31.

4. The method recited in claim 3, wherein the Ala residue is residue 29, 32, 34, and/or 35.

5. The method recited in claim 9, wherein the functional side-chain necessary for the interaction with the N-intein is an Asp, Asn, or Gln residue.

6. The method recited in claim 5, wherein the Asp residue is residue 17 and/or 23.

7. The method recited in claim 5, wherein the Asn residue is residue 25, 30 and/or 36.

8. The method recited in claim 5, wherein the Gln residue is residue 13 and/or 22.

9. The method recited in claim 1, wherein the split C-intein/N-intein is has a structure equivalent to the DnaE intein from Synechocystis sp. PCC6803.

10. The method recited in claim 1, wherein the surface is gold or Si-based.

11. The method recited in claim 1, further comprising:
using UV-light to remove the 2-nitrobenzyl-based protecting group in order to activate the immobilized the C-intein polypeptide.

12. The method recited in claim 1 1, wherein the source of UV-light is a 10 μW pulse of a 354-nm UV light.

13. The method recited in claim 9, wherein the surface is gold or Si-based.

14. The method recited in claim 9, further comprising:
using UV-light to remove the 2-nitrobenzyl-based protecting group in order to activate the immobilized the C-intein polypeptide.

15. The method recited in claim 14, wherein the source of UV-light is a 10 μW pulse of a 354-nm UV light.
